(19) United States
(12) Patent Application Publication
(10) Pub. No.: US 2025/0022598 A1
(43) Pub. Date: Jan. 16, 2025

(54) INFORMATION PROCESSING SYSTEM, INFORMATION PROCESSING METHOD, AND PROGRAM

(71) Applicant: MITSUBISHI TANABE PHARMA CORP, Osaka-shi (JP)

(72) Inventors: Toyokazu SAIKI, Osaka (JP); Yoshito NAGANO, Osaka (JP)

(73) Assignee: MITSUBISHI TANABE PHARMA CORP, Osaka-shi (JP)

(21) Appl. No.: 18/715,127

(22) PCT Filed: Dec. 1, 2022
(86) PCT No.: PCT/JP2022/044439

§ 371 Date: May 31, 2024

(30) Foreign Application Priority Data
Dec. 3, 2021 (JP) 2021-197223

(51) Int. Cl. G16H 50/20; A61B 3/02; G16H 10/60

(52) U.S. Cl. CPC G16H 50/20; A61B 3/02; G16H 10/60

(57) ABSTRACT

[Problem] For easily discovering a disease status of NMOSD, and early detecting a recurrence sign from patient input data in the future, it is possible to provide the information processing system for visualizing a disease-related information on an optic nerve disease including a neuromyelitis optica spectrum disorder.

[Solution] An information processing system of the present invention, comprises: a disease-related information acquisition part acquires a disease-related information including at least an optic nerve disease-related information of a subject; a visualization information addition part adds a visualization information generated based on the disease-related information including the optic nerve disease-related information to a first figure that representing a shape of at least a part of a human body; and a display information generation part generates a display information including a second figure that the visualization information is added to the first figure.

### TECHNICAL FIELD

The present invention relates to an information processing system, an information processing method, and a program for visualizing a disease-related information on an optic nerve disease including a neuromyelitis optica spectrum disorder.

### BACKGROUND TECHNOLOGY

Neuromyelitis optica spectrum disorder (hereinafter referred to as “NMOSD”) is a prevalence in Japan is that said to be 2 to 4 per 100,000 people and an autoimmune disease of a central nervous system that can be fatal by repeated a severe recurrence. An immune system of a body attacks a healthy cell (commonly in an optic nerve, a spinal cord, and a brain), as a result, leading to a recurrence and a severe injury, as a result, it may cause an eye pain and a blindness, a severe muscle weakness, a paralysis, a numbness, a decreased function of an intestine and a bladder, or respiratory failure.

For instance. Patent Document 1 describes an invention related to an anti-inflammatory preparation that improves or alleviates a symptom of the neuromyelitis optica.

### Prior Art List

### Patent Document

Patent document 1: Japanese Unexamined Patent Application Publication No. 2016-540746

### SUMMARY

### Technical Problem

As mentioned above, the NMOSD is a disorder with repeated severe recurrences. However, patients often do not adequately inform their doctors of symptoms that may be related to recurrence at the time of a medical treatment, and both patients and doctors have concerns. Therefore, there is a need for a digital tool that can provide doctors with quantitative and/or qualitative information about patients during their medical treatment days and enable early discovery of any sign of recurrence of NMOSD.

An object of the present invention is to provide an information processing system for visualization of disease-related information of the optic nerve disease, including the neuromyelitis optica spectrum disorder, in order to enable communication of information such as daily NMOSD-related symptoms from a patient to a doctor and, in the future, early discovering a recurrence sign from patient-input data.

### Technical Solution

The present invention for solving the above problems, comprises: a disease-related information acquisition part acquires a disease-related information including at least an optic nerve disease-related information of a subject: a visualization information addition part adds a visualization information generated based on the disease-related information including the optic nerve disease-related information to a first figure that representing a shape of at least a part of a human body; and a display information generation part generates a display information including a second figure that the visualization information is added to the first figure.

Other, the present application discloses a problem and a method for solving will be clarified by a section of embodiments of an invention and drawings.

### Advantageous Effects

According to the present invention, for easily discovering the recurrence sign of the NMOSD, it is possible to provide the information processing system for visualizing the disease-related information including the optic nerve disease-related information.

### BRIEF DESCRIPTION OF THE DRAWINGS

FIG. 1 is a diagram showing an overall configuration instance of an information processing system according to the present embodiment.

FIG. 2 is a diagram showing a hardware configuration instance of a server device 10.

FIG. 3 is a diagram showing a software configuration instance of a server device 10.

FIG. 4 is a diagram showing a configuration instance of a subject information storage part 131.

FIG. 5 is a diagram showing a configuration instance of a disease-related information storage part 132.

FIG. 6 is a diagram showing an instance of FIG. 2 included in a display information.

FIG. 7 is a diagram showing a flow of processing executed in an information processing system of the present embodiment.

### DETAILED DESCRIPTION OF THE PREFERRED EMBODIMENT

Contents of embodiments of the present invention will be listed and explained. One embodiment of the present disclosure comprises the following configuration.

### [Item 1]

An information processing system, comprises:
a disease-related information acquisition part acquires a disease-related information including at least an optic nerve disease-related information of a subject;
a visualization information addition part adds a visualization information generated based on the disease-related information including the optic nerve disease-related information to a first figure representing a shape of at least a part of a human body; and
a display information generation part generates a display information including a second figure that the visualization information is added to the first figure.

### [Item 2]

The information processing system according to item 1, wherein
the optic nerve disease related information includes at least a part of a test result information of a test using a flicker stimulation for the subject.

### [Item 3]

The information processing system according to item 2, further comprising a disease test part provides the test using the flicker stimulation.

### [Item 4]

An information processing method, comprises:
a step of, by a disease-related information acquisition part, acquiring a disease-related information including at least an optic nerve disease-related information of a subject;
a step of, by a visualization information addition part, adding a visualization information generated based on the disease-related information including the optic nerve disease-related information to a first figure that representing a shape of at least a part of a human body; and
a step of, by a display information generation part, generating a display information including a second figure that the visualization information is added to the first figure is characterized.

### [Item 5]

A program realizing on a computer, comprises:
a disease-related information acquisition part acquires a disease-related information including at least an optic nerve disease-related information of a subject;
a visualization information addition part adds a visualization information generated based on the disease-related information including the optic nerve disease-related information to a first figure that representing a shape of at least a part of a human body; and
a display information generation part generates a display information including a second figure that the visualization information is added to the first figure.

### [Item 6]

A computer-readable storage medium storing a program for causing a computer to execute a process, comprising:
acquiring a disease-related information including at least an optic nerve disease-related information of a subject, by a disease-related information acquisition part;
adding a visualization information generated based on the disease-related information including the optic nerve disease-related information to a first figure that representing a shape of at least a part of a human body, by a visualization information addition part; and
generating a display information including a second figure that the visualization information is added to the first figure, by a display information generation part.

Hereinafter, the embodiments of the present disclosure will be described with reference to the drawings. Note that, in this specification and the drawings, about components having substantially a same functional configuration, by designating the same reference numerals, redundant explanation will be omitted.

### <System Configuration>

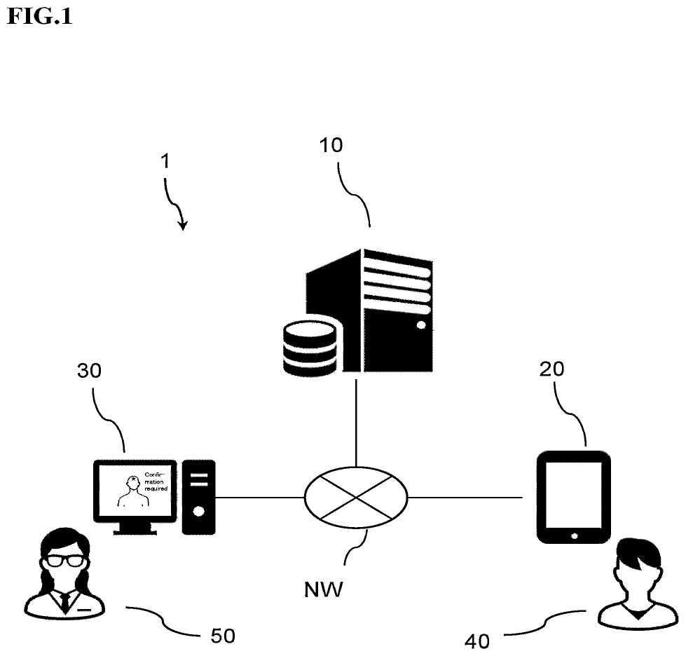

FIG. 1 is a diagram showing a configuration instance of the information processing system (hereinafter also referred to as “this system”) according to an embodiment of the present disclosure. A present system displays on the user terminal (for instance, a doctor terminal) that the second figure that adding the visualization information generated based on the disease-related information that includes at least the optic nerve disease information of the subject (for instance, including at least a part of the test result information of the flicker test performed by an application executed on a subject terminal) to the first figure that representing a shape of at least a part of the human body.

FIG. 1 is a diagram showing an instance of an overall configuration of the information processing system according to the present embodiment. As shown in FIG. 1, about an information processing system 1 according to the present embodiment is, a server device 10, a subject terminal 20, and a user terminal 30 are connected to each other so as to be able to communicate with each other via a communication network NW. The communication network NW is, for instance Internet or a LAN (Local Area Network), and is constructed by a public telephone line network, a private telephone line network, a mobile phone line network. Ethernet (registered trademark), a wireless communication path, or the like.

The server device 10 is a computer that manages the entire information processing system. The server device 10 is, for instance, a workstation, a personal computer, a virtual computer logically realized by a cloud computing, or the like.

The subject terminal 20 is a device owned by a subject 40, who is a target of the confirmation of an information related to a disease (hereinafter referred to as the “disease-related information”) by a user 50 (for instance, a doctor or the like), and is included a device such as a smartphone, the personal computer, or a tablet terminal or the like. Note that the subject terminal 20 can be installed with an application program, a arithmetic device of the subject terminal 20 can execute all or part of functions executed by the server device 10, and a storage part built into the subject terminal 20 may store all or part of the information stored in a storage device 103 that the server device 10 comprising. And conversely, the arithmetic device of the server device 10 may execute all or part of the functions executed by the subject terminal 20, and the storage part built into the server device 10 may store all or part of information stored in the storage device 103 that the subject terminal 20 comprising.

The user terminal 30 is a device that a user (for instance, the doctor) confirm the disease-related information of the subject, and is included the personal computer, the smartphone, the tablet terminal, or the like. Note that the user terminal 30 can be installed with an application program, a arithmetic device of the user terminal 30 can execute all or part of the functions executed by the server device 10, and a storage part built into the user terminal 30 may store all or part of the information stored in the storage device 103 that the server device 10 comprising. And conversely, the arithmetic device of the server device 10 may execute all or part of the functions executed by the user terminal 30, and the storage part built into the server device 10 may store all or part of the information stored in the storage device 103 that the user terminal 30 comprising.

Next, details of a configuration of the information processing system 1 will be explained.

### <Hardware Configuration>

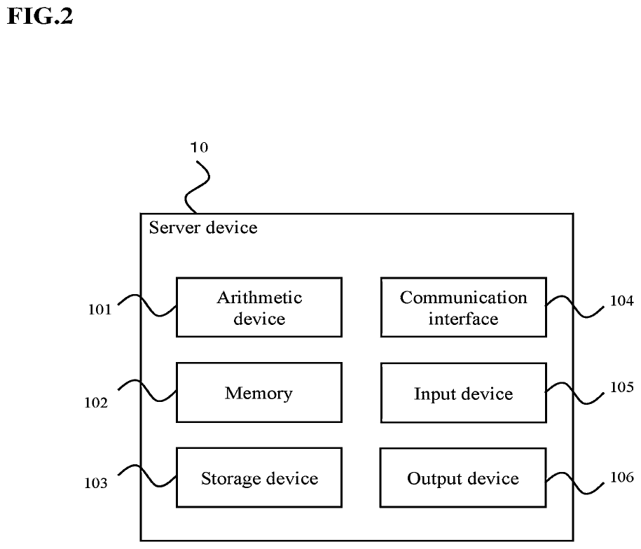

FIG. 2 is a diagram showing a hardware configuration instance of the computer that realizes the server device 10 in the present embodiment. The server device 10 comprises an arithmetic device 101, a memory 102, the storage device 103, a communication interface 104, an input device 105, and an output device 106. These are electrically connected to each other through a bus (not shown). Note that, as described above, the subject terminal 20 or the user terminal 30 or other devices may comprise or execute the devices such the storage device 103 or the like that included in the server device 10, or all or part of the functions executed by the arithmetic device 101.

The arithmetic device 101 controls an overall operation of the server device 10, controls a transmission and reception of a data between each an element, and performs an information processing or the like necessary for an application execution and an authentication processing. The arithmetic device 101 is, for instance, a processor such as a CPU (Central Processing Unit) or a GPU (Graphics Processing Unit) or the like, and by executing a program or the like that stored in the storage device 103 and developed in the memory 102, performs the various information processing.

The memory 102 includes a main memory configured with a volatile storage device such as a DRAM (Dynamic Random Access Memory) or the like, and an auxiliary memory configured with a non-volatile storage device such as a flash memory or an HDD (Hard Disc Drive). The memory 102 is used as a work area or the like of the arithmetic device 101. The memory 102 may also store a BIOS (Basic Input/Output System) that is executed when the server device 10 is started up, and a various setting information or the like.

The storage device 103 stores the various data and the various programs such as the application programs or the like. The storage device 103 is, for instance, the hard disk drive, a solid-state drive, the flash memory, or the like.

The communication interface 104 is an interface for connecting the server device 10 to the communication network NW, and is for instance, an adapter for connecting to Ethernet (registered trademark), a modem for connecting to the public telephone line network, and a wireless communication device for performing a wireless communication, a USB (Universal Serial Bus) connector for a serial communication, a RS132C connector, or the like. The server device 10 is connected to the network NW via the communication interface 104.

The input device 105 is, inputting the data, for instance, a keyboard, a mouse, a touch panel, a button, a microphone, a camera (an imaging part), or the like.

The output device 106 is, outputting the data, for instance, a display, a printer, a speaker, or the like.

### <Software Configuration>

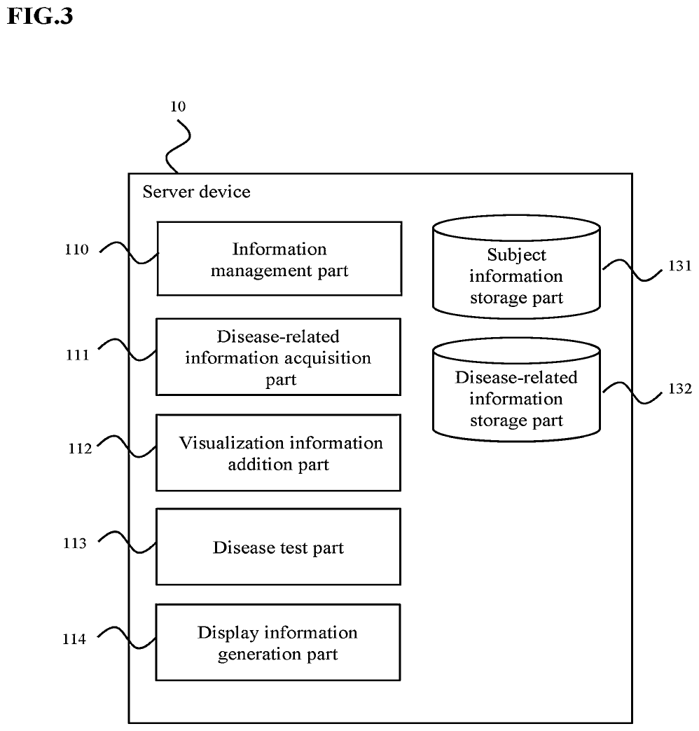

FIG. 3 is a diagram showing a software configuration instance of the server device 10 in the present embodiment. As shown in FIG. 3, the server device 10 comprises each functional parts such as an information management part 110, a disease-related information acquisition part 111, a visualization information addition part 112, a disease test part 113, a display information generation part 114, and each storage parts such as a subject information storage part 131 and a disease-related information storage part 132. Note that, if necessary, the server device 10 may comprise a user information storage part (not shown) stores the information (for instance, a user ID, an affiliation, a contact information, or the like) regarding the user (for instance, the doctor).

Note that, each of the above functional parts are realized by the arithmetic part 101 that the server device 10 comprising, reads and executes the program stored in the storage device 103 into the memory 102, each of the above storage parts are realized as part of the memory 102 that the server device 10 comprising and a storage area provided by the storage device 103.

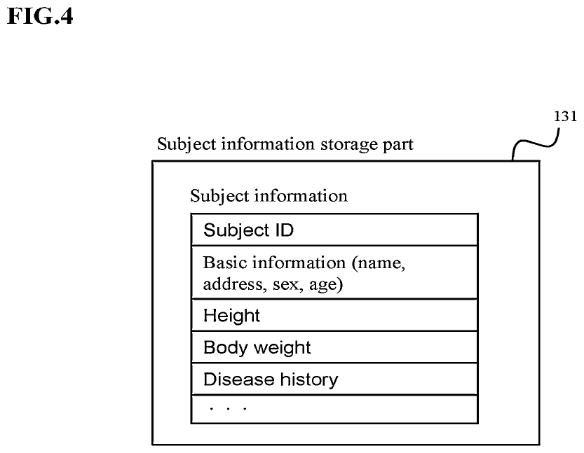

The subject information storage part 131 stores the information regarding the subject 40. FIG. 4 is a diagram showing a configuration instance of the subject information storage part 131. As shown in FIG. 4, the subject information that stored by the subject information storage part 131 includes, in association with a subject ID that identifies the subject 40, a basic information (a personal information such as a name, an address, a sex, an age, or the like), a physical information such as a height and a body weight or the like, and the information such as a medical history and a progression or the like related to the optic nerve disease (particularly, the NMOSD).

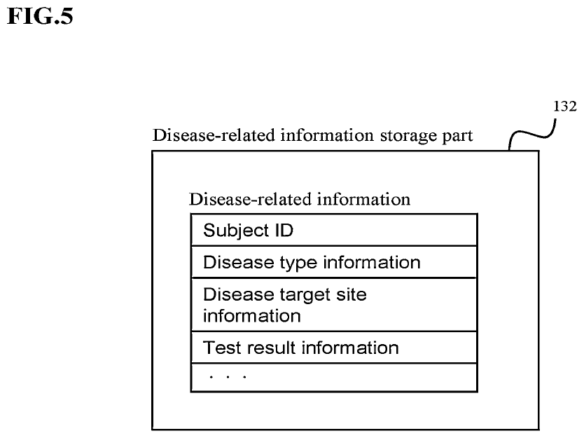

The disease-related information storage part 132 stores the disease-related information including the information related to a various disease such as the test result information regarding the various disease of the subject 40. FIG. 5 is a diagram showing the configuration instance of the disease-related information storage part 132. As shown in FIG. 5, the disease-related information storage part 132 stores the disease-related information, and the disease-related information includes, in association with a subject ID that identifies the subject, a disease type information indicating a type of the disease that the subject is suffering from, linked to each the disease type information, a disease target site information indicating a target body site that has developed that the disease that the subject is suffering from, linked to each the disease type information, a test result information for the disease that the subject is suffering from. In present embodiment, the subject information storage part 131 and the disease-related information storage part 132 are illustrated as being configured separately from each other for convenience, however, all or part of the information included in the disease-related information storage part 132 may be integrated into the subject information storage part 131.

The disease-related information is the information related to the various diseases of the subject 40, and, relation to particularly the NMOSD, includes the optic nerve disease-related information. The disease-related information may be, for instance, the test result information generated by the subject 40 performing a test for the various diseases using the application (including a web browser) executed on the subject terminal 20. The optic nerve disease-related information may be, for instance, test result information of a flicker test. The test result information may be the information on a result of the test provided from the disease test section 113 of the server device 10 to the subject terminal 20, or may be, in cooperation with an external application of the present system, the information that acquiring on the results of performing the test provided from the external application, or may be acquired through the subject terminal 20, or may be acquired by direct access from the server device 10.

The information management part 110 reads a predetermined information from the various storage parts and transmits to an external (for instance, the subject terminal 20 or the user terminal 30), and stores the predetermined information receiving from the external in the corresponding storage part. As a more specific instance, for instance, corresponding to a request transmitted from the user terminal 30 by a user operation, the information management part 110 may read the predetermined information from the subject information storage part 131 and/or the disease-related information storage part 132 or the like and send to the user terminal 30, may be receive the test result information generated by the application (including the web browser) executed on the subject terminal 20, and may be stored in the disease-related information storage part 132.

The disease-related information acquisition part 111 acquires, corresponding to the request from the user terminal 30, the disease-related information (particularly, the optic nerve disease-related information) stored in the disease-related information storage part 132.

The visualization information addition part 112 adds the visualization information generated based on the disease-related information (particularly, the optic nerve disease-related information) to the first figure representing the shape of at least a portion of the human body. The first figure that added to the visualization information is called the second figure. The generated visualization information may have a different form corresponding to a disease degree information determined based on the disease-related information. The disease degree information is, for instance, when the disease-related information includes the test result information may be the information expressed a disease degree indicated in the test result information (for instance, the information or the like regarding the disease degree expressed as A to F as the test result), or may be the information indicated that a degree of a disease corresponding to a comparison result between a numerical value indicated in the test result information and a predetermined reference value (for instance, a disease degree classification information representing a classification corresponding to the comparison result, in which the classification indicates the degree of disease based on predefined reference values). The classification may be, same as the test results, an ordered string of characters such as A-F or I-V. or may be a string with a graduated ordered string of characters meaning, such as safety-caution-danger, these are not limited, and may be set by an administrator or the user of the server device 10 as appropriate.

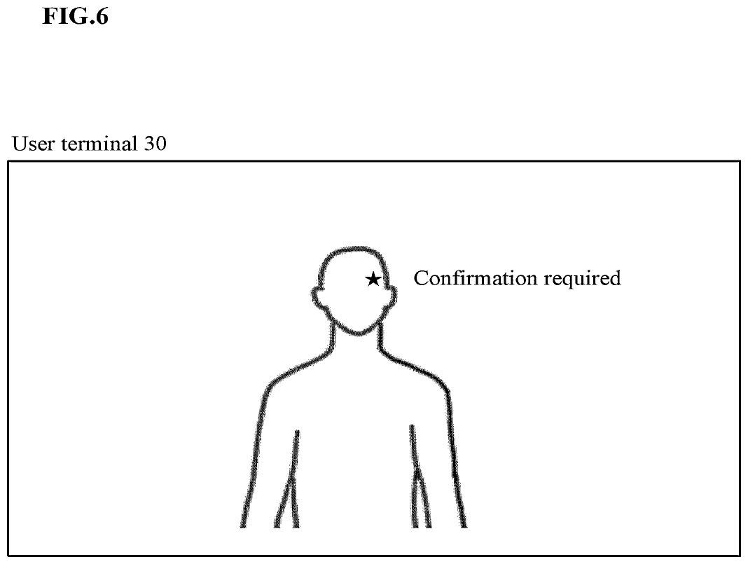

The visualization information may be included, for instance, as shown in FIG. 6, a graphic (a star mark in FIG. 6) indicating a position of the disease based on the disease type information and/or the disease target site information. And, for instance, corresponding to the degree of the disease indicated by the disease degree information, a size and/or a shape of the graphic may differ (for instance, the size will be larger if the degree of the disease is severe, a circle if it is mild, a star if it is severe, or the like), or the color of the graphic may be different (for instance, the color may be blue if the disease is mild, red if the disease is severe, or the like). In addition, the visualization information may be represented in the first figure, instead of using the graphic, by coloring at the location of the disease, or by coloring in a so-called heat map with multiple colors based on the disease degree information (for instance, colorless or white if there is no disease, blue if the disease is mild, red if the disease is severe, or the like).

Further, the visualization information may be included a text information provided near the location of the disease based on either the disease type information and/or the disease target site information, or may be included the text information selected from predetermined text information (for instance, the text information prompting a confirmation such as “confirmation required” as illustrated in FIG. 6) stored in advance in the server device 10, a content of the text information may be changed based on particularly the disease degree information (for instance. “follow-up an observation”. “confirmation required”, or the like). Furthermore, the visualization information may be included the text information indicating a disease name based on the disease type information, or may be included the text information that refers to the information included in the test result information (for instance, a numerical value of the test result, the degree of the disease, or the like). Note that, as shown in FIG. 6, the graphic and the text information may be displayed in combination.

The disease test part 113 is, for acquiring the above-mentioned test result information, a provided functional part when the server device 10 provides a test method, and provides the test corresponding to the various diseases. The test may be, for instance, a test using the flicker stimulation (for instance, stimulation or the like by a flashing light) such as a flicker test. For example, the flicker test may be a test in which blinking light (the blinking light is the flicker stimulation, for instance, a pixel at a predetermined position displayed on the subject terminal 20) is presented to the subject 40 on the subject terminal 20 while increasing or decreasing a blinking frequency of the blinking light, or changing a difference in brightness (contrast) between when the blinking light is high brightness and when the blinking light is low brightness. The disease test part 113 may store a setting information (for instance, the blinking frequency, a switching frequency, a brightness value that each of a high and a low when switching, or the like) in the disease-related information storage part 132 as the test result information in response to the input operation by the subject 40 when changing the status of the blinking light as the flicker stimulation (when the blinking becomes visible or invisible). Note that when a predetermined test is executed by the application on the subject terminal 20, the arithmetic processing part of the subject terminal 20 may be comprised the part or all of function of the disease test section 113.

The display information generating part 114 generates the display information (including the second figure that the visualization information is added to the first figure) for displaying a predetermined display on the display part of the subject terminal 20. Note that when the predetermined display is controlled by the application on the subject terminal 20 rather than a web service via a web browser, the arithmetic processing part of the subject terminal 20 may be comprising the part or all of function of the display information generation part 114.

In this way, particularly, by displaying on the user terminal 30, the second figure that the visualization information generated based on the disease-related information including the optic nerve disease-related information based on the flicker test is added to the first figure that representing the shape of at least a part of the human body, the user 50 such as the doctor can easily discover the disease status of the NMOSD. In the future, early detection of recurrence signs will be possible from the data input by the patient.

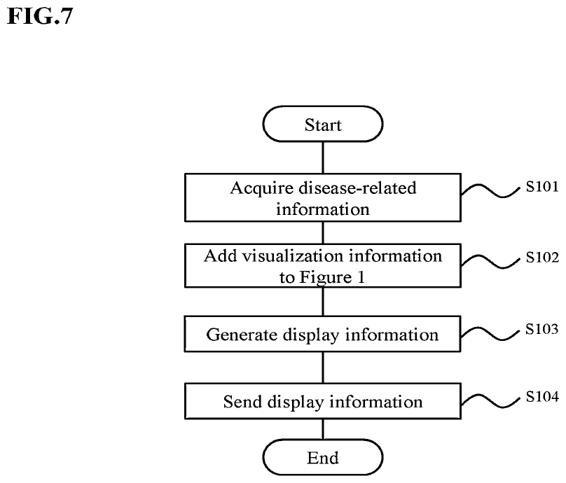

FIG. 7 is a diagram showing the flow of a processing executed in the information processing system of the present embodiment.

First, as pre-processing for the present processing, the information management part 110 of the server device 10 receives the information input regarding the subject 40 (for instance, a patient), and stores the subject information in the subject information storage part 131. The subject information may be input via the user terminal 30 by the user 50 or may be input via the subject terminal 20 by the subject 40.

In addition, as a pre-processing of the present processing, the subject 40 performs or the like the tests (particularly, the flicker test) related to the various diseases on the subject terminal 20, and stores the disease-related information (particularly, the optic nerve disease-related information) in the storage part 132.

Next, in step S101, the disease-related information acquisition part 111 acquires, corresponding to the request from the user terminal 30, the disease-related information (particularly, the optic nerve disease-related information) stored in the disease-related information storage part 132.

Next, in step S102, the visualization information adding part 112 adds the visualization information generated based on the disease-related information (particularly, the optic nerve disease-related information) to the first figure that representing the shape of at least a part of the human body.

Next, in step S103, the display information generation part 114 generates, for displaying a predetermined display on the display part of the subject terminal 20, the display information (including the second figure that visualization information is added to the first figure).

Next, in step S104, the information management part 110 transmits the display information generated that corresponding to the request from the user 50, to the user terminal 30.

As described above, according to the present embodiment, for easily discover the recurrence signs of the NMOSD, it is possible to provide the information processing system for visualizing the disease-related information including the optic nerve disease-related information.

Hereinafter, variations of the information processing system of the above embodiment will be described. In the above information processing system, the server device 10, the subject terminal 20, and the user terminal 30 are connected to each other so as to be able to communicate with each other via a communication network NW, and a third-party terminal (not shown) owned by a third party different from the subject 40 and the user 50 may be also connected via the communication network NW. The third-party terminal is a device owned by the third party and includes a personal computer, smartphone, tablet terminal, or the like. The third party may be, for instance, a subject-related person such as a family member or caregiver of the subject 40, an external organization-related person such as an employee of a medical device company, an employee of a pharmaceutical company, or an employee of a contracted drug development organization a clinical trial conductor.

According to this variation, the third-party terminal may also receive at least one of the above disease-related information (in particular, optic nerve disease-related information), or the second figure in which the above visualization information is added to the first figure representing the shape of at least a part of the human body, and may display the received information for the third party so as to be able to check.

More specifically, for example, by executing a process to associate the subject ID of the subject 40 with the subject-related person ID of the subject-related person such as the family member or caregiver of the subject 40 in an application executed on the subject terminal 20 or user terminal 30, the disease-related information of the subject 40 or the second figure may be received at the third-party terminal, so that the subject-related person may also check the disease status of the subject 40. As a result, for instance, when the subject 40 becomes a person in need of care, the disease status can be easily confirmed by the caregivers of the subject 40.

For example, if the third party is the external organization-related person such as an employee of a medical device company, by executing a viewing permission process for the external organization-related person ID of the external organization-related person in an application executed on the subject terminal 20 or user terminal 30, the disease-related information or the like regarding authorized the subject 40 may be received at the third party terminal of the external organization-related person, so that the external organization-related person may also check the disease status of authorized the subject 40. In this case, information (for instance, information anonymized by the server device 10, or the like) that does not include at least one of the names or addresses of authorized the subject 40 may be sent to the third-party terminal of the external organization-related person. As a result, various information about the disease can be easily collected even by persons from external organization who need the various information about the disease for research or other purposes. In particular, if the information is configured to be transmitted without including at least one of the names or addresses of authorized the 40 subjects, it is possible to ensure anonymity and the like.

Note that the above information processing system is also applicable to other a neurological disease such as multiple sclerosis (MS) or the like.

That is, it may be the information processing system, comprises:
the disease-related information acquisition part acquires the disease-related information including at least the optic nerve disease-related information of the subject;
the visualization information addition part adds the visualization information generated based on the disease-related information including the optic nerve disease-related information to the first figure that representing the shape of at least a part of the human body; and
the display information generation part generates the display information including the second figure that the visualization information is added to the first figure.

### Description of the Reference Numerals

1 Information processing system
10 Server device
20 Subject terminal
30 User terminal
40 Subject
50 User
NW Communication network

1. An information processing system, comprising:
circuitry configured to acquire a disease-related information including at least an optic nerve disease-related information of a subject, add a visualization information generated based on the disease-related information including the optic nerve disease-related information to a first figure that representing a shape of at least a part of a human body, and generate a display information including a second figure that the visualization information is added to the first figure,
wherein the disease-related information includes a disease type information, a disease target site information, and a test result information for a disease, the visualization information includes a graphic indicating a position of the disease based on the disease-related information, and the circuitry is configured to add the graphic having a different form corresponding to a disease degree information determined based on the disease-related information to the first figure.

2. The information processing system according to claim 1, wherein the optic nerve disease-related information includes at least a part of a test result information of a test using a flicker stimulation for the subject.

3. The information processing system according to claim 2, wherein the circuitry is configured to provide the test using the flicker stimulation.

4. An information processing method, comprising:
acquiring a disease-related information including at least an optic nerve disease-related information of a subject;
adding a visualization information generated based on the disease-related information including the optic nerve disease-related information to a first figure that representing a shape of at least a part of a human body; and
generating a display information including a second figure that the visualization information is added to the first figure,
wherein the disease-related information is acquired by using an information processing system comprising circuitry configured to acquire the disease-related information, the visualization information is added by using the information processing system comprising the circuitry configured to add the visualization information, the display information is generated by using the information processing system comprising the circuitry configured to generate the display information, the disease-related information includes a disease type information, a disease target site information, and a test result information for a disease, the visualization information includes a graphic indicating a position of the disease based on the disease-related information, and the circuitry is configured to add the graphic having a different form corresponding to a disease degree information determined based on the disease-related information to the first figure.

5. A non-transitory computer-readable medium storing a program that when executed by a computer, an information processing system is caused to execute a method comprising:
acquiring a disease-related information including at least an optic nerve disease-related information of a subject;
adding a visualization information generated based on the disease-related information including the optic nerve disease-related information to a first figure that representing a shape of at least a part of a human body; and
generating a display information including a second figure that the visualization information is added to the first figure,
wherein the disease-related information is acquired by using an information processing system comprising circuitry configured to acquire the disease-related information, the visualization information is added by using the information processing system comprising the circuitry configured to add the visualization information, the display information is generated by using the information processing system comprising the circuitry configured to generate the display information, the disease-related information includes a disease type information, a disease target site information, and a test result information for a disease, the visualization information includes a graphic indicating a position of the disease based on the disease-related information, and the circuitry is configured to add the graphic having a different form corresponding to a disease degree information determined based on the disease-related information to the first.

6. The information processing system according to claim 3, wherein the circuitry is configured to generate the test result information including a setting information in response to an input operation by the subject when the flicker stimulation is changed.

7. The information processing system according to claim 1, wherein the circuitry ins configured to transmit the display information indicating a disease status of the subject with respect to a neuromyelitis optical spectrum disorder based on the second figure.
